(19) United States
(12) Patent Application Publication
(10) Pub. No.: US 2022/0233352 A1
(43) Pub. Date: Jul. 28, 2022

(54) OPHTHALMIC CANNULA WITH EXTERNAL ENGAGEMENT FEATURE

(71) Applicant: Medical Instrument Development Laboratories, Inc., San Leandro, CA (US)

(72) Inventor: Erik W. Peterson, Walnut Creek, CA (US)

(21) Appl. No.: 17/157,672

(22) Filed: Jan. 25, 2021

(51) Int. Cl. A61F 9/007

(52) U.S. Cl. CPC A61F 9/00736; A61F 9/00781

(57) ABSTRACT

An ophthalmic surgical system includes a cannula having a receptacle. The receptacle includes a valve and a first lip extending inwardly and defining at least a portion of a chamber in the receptacle. The ophthalmic surgical system also includes a surgical instrument having a second lip that is sized and shaped to engage with the first lip of the cannula to couple the surgical instrument to the cannula.

### BACKGROUND

Ophthalmic surgeries require that a portion of a surgical instrument must pass through the wall of the eye and into the interior of the eye. A simple incision through the wall of the eye can suffice to provide such a passageway. In modern practice, however, an ophthalmic cannula is usually placed into this incision. The cannula provides a rigid tubular passageway through the wall of the eye. During some surgeries, three ophthalmic cannulae are placed at various locations in the wall of the eye. Two of these facilitate the passage of surgical instruments held in the surgeon's two hands. The third provides an attachment point for an infusion tube, which delivers fluid into the eye to replace material removed through the surgical instruments and to maintain the fluid pressure inside the eye.

Ophthalmic surgical instruments typically include a handle portion and a needle portion. The needle portion comprises a cylindrical tube. The distal end of the tube incorporates functional elements such as cutting blades or forceps tips, and the mechanisms for operating these functional elements pass through the interior of the tube. The needle portion of the surgical instrument passes through the ophthalmic cannula into the interior of the eye. In order to place the functional elements at the tip of the needle at the location in the eye where the surgical function is to be performed, it is necessary to insert the needle through the cannula to various depths and to rotate the needle (and with it the cannula) to various angles with respect to the wall of the eye. These manipulations can place a stress on the needle, and on the mechanisms disposed therein, particularly at the location where the needle enters the ophthalmic cannula.

With the trend toward smaller needle sizes (from approximately 0.9 mm diameter for earlier instruments to approximately 0.4 mm diameter for more recent instruments) instrument failure due to needle bending stresses is increasingly common. One solution to this problem is proposed in U.S. Pat. No. 10,179,007, the entire contents of which are incorporated by reference herein. The surgical instrument includes a reinforcing slider coaxial with the needle. The reinforcing slider is movable with respect to the handle and the needle, such that the position of the reinforcing slider may be adjusted to vary the length of the portion of the needle that extends past the slider end portion. It is intended that the slider end portion be in contact with the cannula during use, such that movements of the instrument handle to cause rotation of the needle and the cannula with respect to the wall of the eye are communicated to the cannula through the reinforcing slider rather than through the needle.

### SUMMARY

The present disclosure relates, for example, to engagement features incorporated into an ophthalmic cannula which facilitate communication of forces from a reinforcing slider to the cannula. These engagement features may also be used to facilitate attachment of an infusion tube to the cannula. While current cannula systems also have engagement features for attachment of an infusion tube, the structures of the systems described herein are unique (at least in part) in the location of the engagement features relative to other features such as a valve.

Specifically, the systems described herein relate for example to structures in which an engagement feature(s) is disposed at or near the end of a cannula opposite an end which enters the eye. If a valve is present in such a structure, it is disposed between the engagement feature(s) and an interior of the cannula.

In accordance with one example embodiment, an ophthalmic surgical system includes a cannula having a receptacle or a cannula closure. The receptacle includes a valve and a first lip extending inwardly and defining at least a portion of a chamber in the receptacle. The ophthalmic surgical system also includes a surgical instrument having a second lip that is sized and shaped to engage with the first lip of the cannula to couple the surgical instrument to the cannula.

In accordance with another example embodiment, a cannula includes a tubular portion having a hollow interior, and a receptacle coupled to the tubular portion. The receptacle includes an internal chamber disposed at a first end of the receptacle and adjacent to and open to the hollow interior of the tubular portion. The receptacle also includes an external chamber disposed at an opposite end of the receptacle, and a valve disposed between the internal chamber and the external chamber. The valve is movable between a closed position and an opened position. The cannula also includes a lip defining at least a portion of the external chamber. The lip is configured to engage a portion of a surgical instrument.

In accordance with another example embodiment, a method of coupling a surgical instrument to a cannula includes moving a lip of a surgical instrument axially into an external chamber of a receptacle of the cannula, extending a needle of the surgical instrument through a valve in the receptacle, and bending the needle of the surgical instrument until a surface of the lip of the surgical instrument is moved within the exterior chamber and into engagement with a surface of a lip of the receptacle.

Other embodiments and aspects of various embodiments will become apparent by consideration of the detailed description and accompanying drawings.

### BRIEF DESCRIPTION OF THE DRAWINGS

FIG. 1 is a perspective view of an ophthalmic cannula.

FIG. 2 is a partial, enlarged cross-sectional view of a portion of the ophthalmic cannula.

FIG. 3 is a perspective view of a surgical instrument.

FIG. 4 is a partial, enlarged cross-sectional view of a portion of the surgical instrument.

FIG. 5 is a perspective view of an infusion tubing set.

FIG. 6 is a partial, enlarged cross-sectional view of a portion of the infusion tubing set.

FIG. 7 is a cross-sectional view of both the ophthalmic cannula and the surgical instrument during a first assembly step.

FIG. 8 is a cross-sectional view of both the ophthalmic cannula and the surgical instrument during a second assembly step.

FIG. 9 is a cross-sectional view of both the ophthalmic cannula and the surgical instrument during a third assembly step.

FIG. 10 is a cross-sectional view of both the ophthalmic cannula and the infusion tubing set during a first assembly step.

FIG. 11 is a cross-sectional view of both the ophthalmic cannula and the infusion tubing set during a second assembly step.

FIG. 12 is a cross-sectional view of an alternate embodiment of both the ophthalmic cannula and the infusion tubing set during a first assembly step.

FIG. 13 is a cross-sectional view of both the ophthalmic cannula and the infusion tubing set of FIG. 12, during a second assembly step.

FIG. 14 is a cross-sectional view of an alternate embodiment of both the ophthalmic cannula and the surgical instrument during a first assembly step.

FIG. 15 is a cross-sectional view of both the ophthalmic cannula and the surgical instrument of FIG. 14 during a second assembly step.

### DETAILED DESCRIPTION

Before any embodiments are explained in detail, it is to be understood that embodiments are not limited in their application to the details of construction and the arrangement of components set forth in the following description or illustrated in the following drawings. Other embodiments are possible and embodiments described and illustrated are capable of being practiced or of being carried out in various ways.

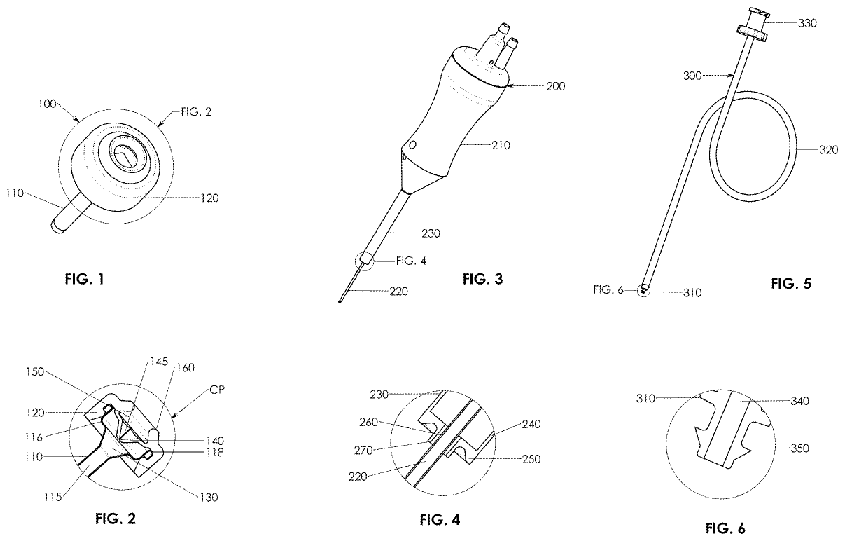

FIGS. 1 and 2 illustrate an example cannula 100. The cannula 100 is described in the context of being used as an ophthalmic cannula, and being used with surgical instruments and/or infusion tubing sets during ophthalmic surgery. The cannula 100, however, may also be used in conjunction with other surgeries and/or procedures, and/or may be coupled to other instruments other than the ophthalmic surgical instruments and infusion tubing sets described herein.

In the example illustrated, the cannula 100 includes a tubular portion 110. The tubular portion 110 may be rigid, and made, for example, at least in part of a hard material (e.g., metal). In some embodiments, the tubular portion 110 includes an angled and/or pointed distal tip, to facilitate insertion through an incision in an eye. As illustrated in FIG. 2, the tubular portion 110 includes a hollow interior 115, to facilitate passage of fluid or other material through the cannula 100 and either into or out of the eye. Other embodiments may include a tubular portion 110 made of a softer and/or flexible material, and/or a tubular portion 110 having a size and shape other than that illustrated.

With continued reference to FIGS. 1 and 2, in some embodiments the tubular portion 110 includes an enlarged proximal end 116 (e.g., generally cup shaped or funnel shaped) with, in one example, a rolled outer periphery 118. The cannula 100 includes a receptacle 120 coupled to (e.g., releasably coupled to, fixed to, or integrally formed as a single piece with) the enlarged proximal end 116 of the tubular portion 110. The receptacle 120, or at least a portion of the receptacle 120, may be softer than the tubular portion 110 (including the enlarged proximal end 116), and made for example at least in part of an elastomeric material. The cannula 100 may slide over or otherwise be coupled to the enlarged proximal end 116. Other embodiments may include different shapes and sizes for a receptacle 120 than that illustrated. In some embodiments the receptacle 120 is co-molded to the end of the tubular portion 110. During use, the receptacle 120 is an external portion of the cannula 100 disposed outside the eye at all times.

With reference to FIG. 2, in the illustrated example embodiment, an internal chamber 130 is disposed within the receptacle 120. The internal chamber 130 is located adjacent to and opens into the hollow interior 115 of the tubular portion 110. In the example illustrated in FIG. 2, the internal chamber 130 has a tapered diameter that extends to the hollow interior 115 of the tubular portion 110. In other embodiments, the internal chamber 130 may have a constant cross-sectional diameter, or a different diameter (e.g. tapered in a different direction) other than that illustrated.

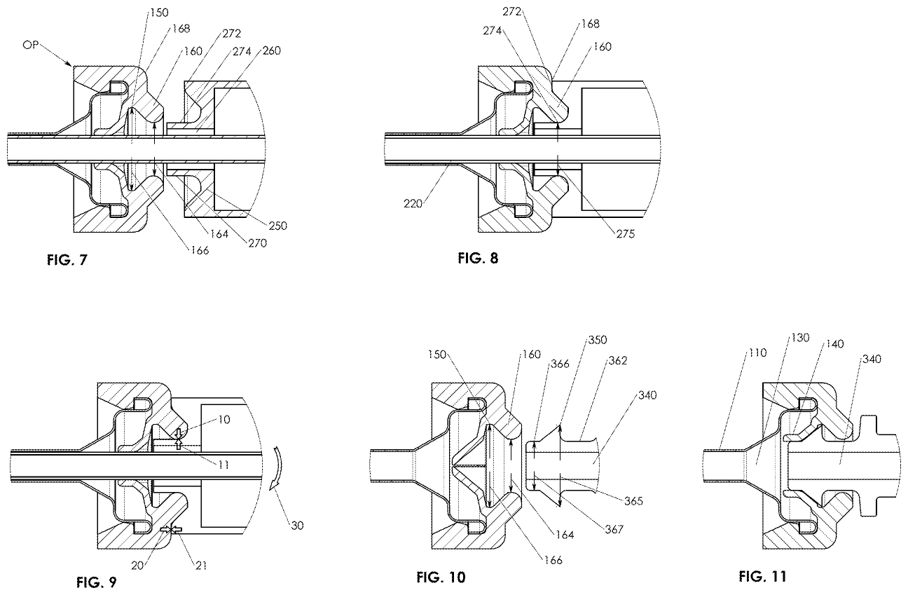

With continued reference to FIG. 2, the receptacle 120 includes a valve 140 within an interior of the receptacle 120. The receptacle 120 receives components but also acts as a closure (particularly when the valve 140 is closed). In the example illustrated, the valve 140 includes at least one flexible membrane or other flexible structure (e.g., a flap or flaps) that define a slit 145. In one example, the slit 145 is positioned in a center of the valve 140. The valve 140 is movable between a closed (e.g., sealed) position and an opened (e.g., unsealed) position. As illustrated in FIG. 2, in the closed position “CP” the slit 145 may be tightly closed such that the valve 140 is sealed. When the valve 140 is in the closed position, fluid and other materials are inhibited or prevented from passing through the slit 145 and the valve 140 from the internal chamber 130 and the tubular portion 110. The valve 140 may thus maintain the fluid pressure inside the eye. With reference to FIGS. 7 and 8, in the opened position “OP” the valve 140 is pushed or otherwise deformed by a surgical instrument or other device (e.g., deformed toward the tubular portion 110). The force from the surgical instrument or other device expands the slit 145 radially outwardly to form an opening to allow a surgical instrument or device to extend through the valve 140. Other embodiments include different shapes and sizes of a valve 140 than that shown.

With reference to FIGS. 1 and 2, the receptacle 120 also includes an external chamber 150. The external chamber 150 is at an opposite side of the receptacle 120 than the internal chamber 130, such that the valve 140 is disposed between the internal chamber 130 and the external chamber 150. The external chamber 150 is disposed adjacent to and is open to the valve 140, such that fluid or other material may flow through the external chamber 150, through the valve 140 (when the valve 140 is opened), through the internal chamber 130, and into the tubular portion 110 (or in a reverse flow).

As illustrated in FIG. 2, the receptacle 120 also includes at least one engagement feature that is used to couple the cannula 100 to another device (e.g., a surgical instrument, an infusion tubing set, etc.). In the illustrated embodiment, the engagement feature includes the external chamber 150, as well as a lip 160. The lip 160 defines at least a portion of the external chamber 150 and is located generally at the end of the receptacle 120. The lip 160 may be a rigid or flexible protrusion or other structure that extends for example radially inwardly, and/or includes a surface or surfaces that facilitate engagement between the cannula 100 and another device (e.g., a surgical device or infusion tubing set). The lip 160 may extend partially or entirely around the external chamber 150 and/or the end of the receptacle 120 (e.g., circumferentially). As illustrated in FIG. 2, the valve 140 is positioned between the lip 160 and the internal chamber 130. The lip 160 protrudes inwardly (e.g., radially inwardly), thus making the external chamber 150 smaller or narrower near the end of the receptacle 120. Other embodiments include different shapes and orientations of the lip 160 than that illustrated. The lip 160 may also be positioned at a location different than the location illustrated.

With reference to FIGS. 3 and 4, the cannula 100 may be coupled to a surgical instrument 200. The surgical instrument 200 may include, for example, a handle 210, a needle 220, and a reinforcing slider 230. In the example illustrated, the reinforcing slider 230 includes a tubular portion 240 and an end portion 250. The end portion 250 includes a central passageway 260. The needle 220 passes through the central passageway 260. In some embodiments, the central passageway 260 is substantially larger than the needle 220. The reinforcing slider 230 is movable with respect to handle 210 and the needle 220, such that the position of the reinforcing slider 230 may be adjusted to vary the length of the portion of the needle 220 that extends past the slider end portion 250.

With continued reference to FIGS. 3 and 4, the surgical instrument 200 also includes at least one engagement feature that is used to couple the surgical instrument 200 to the cannula 100. In the example illustrated, the engagement feature includes a lip 270 disposed at (e.g., extending from, integrally formed as a single piece with, or otherwise coupled to) the end portion 250. The lip 270 extends (e.g., axially and/or radially outwardly from the end portion 250), and as described further below is shaped and sized to engage with at least a portion the lip 160 of the cannula 100. In other embodiments, the lip 270 may have a different shape, orientation, and/or location.

With reference to FIGS. 5 and 6, the cannula 100 may also, or alternatively, be coupled to an infusion tubing set 300. In the example illustrated, the infusion tubing set 300 includes a first connector 310, a flexible tubing 320 coupled to the first connector 310 at one end of the flexible tubing 320, and a second connector 330 coupled to the flexible tubing 320 at a second, opposite end of the flexible tubing 320. The infusion tubing set 300 also includes a central passageway 340 that extends through the first connector 310. In some embodiments, the second connector 330 may be attached to a source of infusion fluid, which is then delivered through tubing 320 and the central passageway 340 of first connector 310.

With continued reference to FIG. 6, the first connector 310 also includes at least one engagement feature that is used to couple the infusion tubing set 300 to the cannula 100. In the illustrated embodiment, the engagement feature includes a lip 350. The lip 350 projects radially outwardly, and defines a barb. As described further below, the lip 350 may engage with at least a portion of the lip 160 of the cannula 100. Other embodiments include different shapes, locations, and orientations of a lip 350 than that illustrated.

With reference to FIGS. 7-9, the surgical instrument 200 may be coupled to the cannula 100 using the engagement feature(s) on the surgical instrument 200 and the cannula 100. For example, in the illustrated embodiment the lip 160 of the cannula 100 is disposed at an entrance to the external chamber 150. The lip 160 projects inwardly, defining an opening. An internal diameter 164 of the opening through the lip 160 is less than an internal diameter 166 of the external chamber 150 adjacent the lip 160. As illustrated in FIG. 7, the lip 160 is also disposed adjacent an external surface 168 at the end of receptacle 120.

With continued reference to FIGS. 7 and 8, in the illustrated example embodiment the surgical instrument 200 includes additional engagement features in the form of an external surface 272 at the end portion 250, and a cylindrical extension 274 projecting from the external surface 272 coaxial with the central passageway 260. As illustrated in FIG. 7, the external surface 272 curves to form a groove or indentation along an area near a base of the cylindrical extension 274, and the lip 270 forms at least a portion of the cylindrical extension 274.

In the illustrated example, a principal diameter 275 of the cylindrical extension 274 is slightly smaller than the internal diameter 164 formed by the lip 160 on the cannula 100, enabling the cylindrical extension 274 to slide into the external chamber 150 with limited or no resistance from the lip 160. In other embodiments, the cylindrical extension 274 may include features with a diameter slightly larger than the internal diameter 164 of a flexible lip 160 so as to provide a slight engagement force between surgical instrument 200 and cannula 100. In general, however, it must be possible to withdraw the surgical instrument 200 from engagement without applying forces which might pull the cannula 100 out of the eye.

When the surgical instrument 200 and the cannula 100 are aligned (and before or after the cylindrical extension 274 has moved into the external chamber 150) the needle 220 of the surgical instrument 200 may be advanced through the external chamber 150 and through the rest of cannula 100, forcing open the valve 140 and advancing into the tubular portion 110. As illustrated in FIG. 8, the external surface 272 of the surgical instrument 200 may come to rest (e.g., be pressed) against the external surface 168 of the cannula 100. In this position, the surgical instrument 200 and the cannula 100 are fully engaged.

In some embodiments, and with reference to FIG. 9, a torsional force 30 may initially bend the needle 220 slightly, forcing the lip 270 to slide up against the lip 160. A torsional force 30 may then be applied to rotate the axis of the cannula 100 without further loading of the needle 220. FIG. 9 illustrates force vectors 10 and 11 applied at an interface of the lip 160 and the lip 270. Note that these are counterbalanced by force vectors 20 and 21 at the interface of the external surfaces 168 and 272. Very large forces can be applied in this manner while protecting the needle 220 from excessive loading. For the purpose of transmitting such large forces, the lip 160 of the cannula 100 may be rigid (e.g., may be made of metal, hard plastic, or other materials). In the illustrated embodiment, however, the lip 160 is elastic, to allow the cannula 100 to also be coupled to the infusion tubing set 300. In some embodiments, the needle 220 may pass through the cannula 100 such that forces of engagement that occur when coupling the surgical instrument 200 to the cannula 100 are directed away from the more delicate portions of the surgical instrument 200.

As shown in FIGS. 10 and 11, the infusion tubing set 300 may also be coupled to the cannula 100 using the engagement features on the infusion tubing set 300 and the cannula 100. In the example illustrated, the engagement features on the infusion tubing set 300 include the lip 350 (e.g., barb) described above, a first cylindrical portion 362, and a second cylindrical portion 366. The lip 350, the first cylindrical portion 362, and second cylindrical portion 366 are coaxially disposed around the central passageway 340. A diameter 367 of the second cylindrical portion 366 is less than the internal diameter 164 defined by the lip 160 on the cannula 100. A diameter 365 of the barbed lip 350 is larger than the internal diameter 164, and in some embodiments may even be slightly larger than the internal diameter 166 of the external chamber 150.

To couple the infusion tubing set 300 to the cannula 100, the second cylindrical portion 366 is advanced into the opening through the lip 160 on the cannula 100 until the barbed lip 350 on the infusion tubing set 300 comes into contact with the lip 160. Force is then applied to press the lip 350 through the elastic lip 160 and on into the external chamber 150. The elastic lip 160 then closes down around the first cylindrical portion 362. The portion of the receptacle 120 forming the external chamber 150 may also be elastic, so a hermetic seal may be formed around the lip 350. When in this engaged position (FIG. 11), the second cylindrical portion 366 extends past the external chamber 150 and forces open the valve 140. Opening of the valve 140 facilitates flow of infusion fluid through the central passageway 340, the valve 140, and the internal chamber 130 and into the tubular portion 110. The infusion of fluid then enters the eye. In this engaged position, the lips 160, 350 remain engaged with one another, making it difficult to easily remove the infusion tubing set 300.

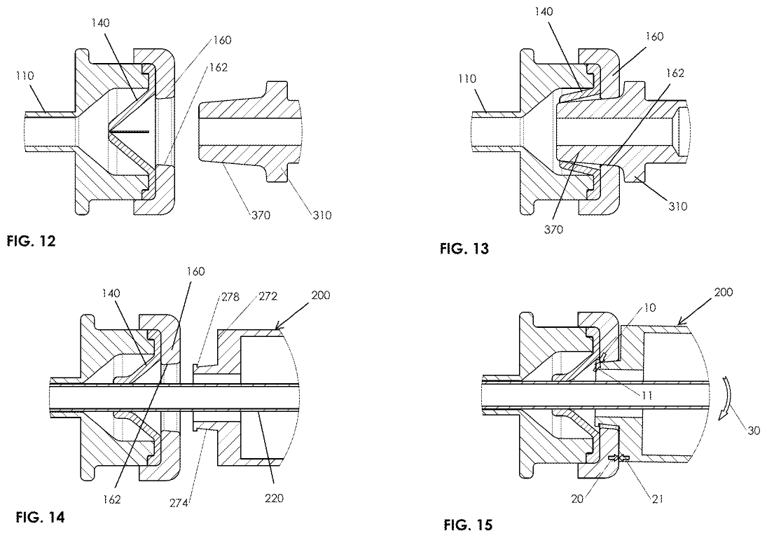

With reference to FIGS. 12 and 13, in some embodiments the lip 160 of the cannula 100 may be rigid, rather than elastic. In the illustrated embodiment the rigid lip 160 is formed as part of a separate piece from the rest of the cannula 100, and extends over a portion of the valve 140, such that the valve 140 is sandwiched between the lip 160 and the rest of the cannula 100. In other embodiments the lip 160 is formed integrally as a single piece with the rest of the cannula 100.

The lip 160 may be formed, for example, with a slight taper on an inner surface 162. In the illustrated embodiment, the tapering of the inner surface 162 defines an internal diameter that decreases moving along a direction toward the tubular portion 110. The first connector 310 may also include a tapered portion 370. In the illustrated embodiment, the tapered portion 370 decreases in diameter moving along a direction toward the tubular portion 110. The inner surface 162 tapers at an identical or nearly identical angle as the tapered portion 370. Other embodiments include different angles and/or tapers for the inner surface 162 and the tapered portion 370 other than that illustrated, as well as different sizes for the lip 160 and the tapered portion 370 other than that illustrated.

With continued reference to FIGS. 12 and 13, when the tapered portion 370 is pressed into the inner surface 162, an interference fit is created which holds the cannula 100 and the infusion tubing set 300 together. In some embodiments, the tapered portion 370 of the first connector 310 is at least partially elastic, such that when the tapered portion 370 is pressed into the inner surface 162, the tapered portion 370 compresses slightly radially inwardly and continues to push radially outwardly, facilitating a tight fit against the rigid lip 160 and the inner surface 162. As illustrated in FIG. 13, once the tapered portion 370 has been inserted into the cannula 100, the valve 140 is forced open, thereby opening a passageway to the tubular portion 110.

With reference to FIGS. 14 and 15, in some embodiments the engagement features of the surgical instrument 200 may include additional features which facilitate use with a cannula 100 having a rigid lip 160. For example, the cylindrical extension 274 projecting from the external surface 272 may include a radially projecting lip 278 (e.g., defined by an undercut at a distal end of the cylindrical extension 274) intended to engage with the rigid lip 160 when a torsional force 30 is applied. Torsional force 30 may initially bend the needle 220 slightly, forcing the cylindrical extension 274 to slide up against the inner surface 162 of lip 160 with projecting lip 278 engaged behind the lip 160. A torsional force 30 may then be applied to rotate the axis of the cannula 100 without further loading of the needle 220. As illustrated in FIG. 15, force vectors 10 and 11 are applied at an interface of the lip 160 and the lip 278, and are counterbalanced by force vectors 20 and 21.

At least one purpose of the engagement features described above is to communicate forces between the cannula 100 and some other object. For example, the engagement features described above may serve the purpose of attaching the infusion tubing set 300 to the cannula 100, and may also serve to communicate forces between the cannula 100 and some portion of the surgical instrument 200, such that the more delicate portions of the surgical instrument 200 are protected from excessive force.

Additionally, and as described above, the lip 160 (and external chamber 150) of the cannula 100 may specifically be located with the valve 140 positioned between the lip 160 (and external chamber 150) and the internal portion of the cannula 100. This is in contrast, for example, to cannulas where engagement features are located instead between a valve and an internal portion of the cannula.

Additionally, and as described above, engagement features in current cannulas are generally of two principal types. The most common type relies on an interference fit between essentially rigid portions of the cannula and an infusion connector. The second type employs an elastic structure in the cannula to engage a rigid infusion connector. The cannula 100 described above employs for example an elastic structure in the cannula 100, such that the cannula 100 may be coupled for example to both the infusion tubing set 300 and to the surgical instrument 200. However, the objectives of the cannula 100 may also be met with a rigid structure.

Although various embodiments have been described in detail with reference to certain examples illustrated in the drawings, variations and modifications exist within the scope and spirit of one or more independent aspects described and illustrated.

What is claimed is:

1. A ophthalmic surgical system comprising:
a cannula having
a tubular portion;
a receptacle coupled to the tubular portion, the receptacle having a first lip projecting inwardly and defining at least a portion of a chamber in the receptacle; and
a surgical instrument having a second lip that is sized and shaped to engage with the first lip of the cannula to couple the surgical instrument to the cannula.

2. The ophthalmic surgical system of claim 1, wherein the chamber is an external chamber, wherein the tubular portion has a hollow interior, wherein the receptacle includes an internal chamber disposed at a first end of the receptacle and adjacent to and open to the hollow interior of the tubular portion.

3. The ophthalmic surgical system of claim 2, wherein the external chamber is disposed at a second, opposite end of the receptacle.

4. The ophthalmic surgical system of claim 3, wherein a valve is disposed between the internal chamber and the external chamber, wherein the valve is movable between a closed position and an opened position.

5. The ophthalmic surgical system of claim 1, wherein the chamber includes a first internal diameter, and wherein the lip defines a second internal diameter that is less than the first internal diameter.

6. The ophthalmic surgical system of claim 5, wherein the second lip of the surgical instrument has a diameter that is less than the second internal diameter.

7. The ophthalmic surgical system of claim 1, wherein the surgical instrument includes a reinforcing slider with an end portion having a central passageway, wherein the second lip extends from the end portion.

8. The ophthalmic surgical system of claim 7, wherein the surgical instrument includes an external surface and a cylindrical extension projecting from the external surface that is coaxial with the central passageway.

9. The ophthalmic surgical system of claim 1, further comprising an infusion tubing set having a first connector, a flexible tubing coupled to the first connector at one end of the flexible tubing, and a second connector coupled to the flexible tubing at a second, opposite end of the flexible tubing, wherein the first connector includes a central passageway and a third lip that is sized and shaped to engage with the first lip of the cannula to couple the infusion tubing set to the cannula.

10. The ophthalmic surgical system of claim 9, wherein the third lip defines a barb.

11. A cannula comprising:
a tubular portion having a hollow interior;
a receptacle coupled to the tubular portion, wherein the receptacle includes
an internal chamber disposed at a first end of the receptacle and adjacent to and open to the hollow interior of the tubular portion,
an external chamber disposed at an opposite end of the receptacle,
a valve disposed between the internal chamber and the external chamber, wherein the valve is movable between a closed position and an opened position, and
a lip defining at least a portion of the external chamber, wherein the lip is configured to engage a portion of a surgical instrument.

12. The cannula of claim 11, wherein the lip projects radially inwardly.

13. The cannula of claim 11, wherein the external chamber includes a first internal diameter, and wherein the lip defines a second internal diameter that is less than the first internal diameter.

14. The cannula of claim 11, wherein the lip is flexible, and is configured to flex when the surgical instrument presses against the lip.

15. The cannula of claim 11, wherein the lip is rigid.

16. A method of coupling a surgical instrument to a cannula, the method comprising:
moving a lip of a surgical instrument axially into an external chamber of a receptacle of the cannula;
extending a needle of the surgical instrument through a valve in the receptacle; and
bending the needle of the surgical instrument until a surface of the lip of the surgical instrument is moved within the exterior chamber and into engagement with a surface of a lip of the receptacle.

17. The method of claim 16, further comprising pressing an external surface of the surgical instrument against an external surface of the receptacle of the cannula.

18. The method of claim 16, further comprising rotating the cannula after the step of bending the needle.

19. The method of claim 16, wherein the step of extending the needle includes extending the needle through an internal chamber of the receptacle of the cannula, and through a tubular portion of the cannula that is coupled to the receptacle.

20. The method of claim 16, further comprising removing the surgical instrument from the cannula and coupling an infusion tubing set to the cannula.
